(19) United States
(12) Patent Application Publication
(10) Pub. No.: US 2006/0266360 A1
(43) Pub. Date: Nov. 30, 2006

(54) Nasal dilator

(75) Inventor: Louis O. Noce, Sanford, FL (US)

Correspondence Address: NIXON & VANDERHYE, PC, 901 NORTH GLEBE ROAD, 11TH FLOOR, ARLINGTON, VA, 22203, US

(73) Assignee: Alisa A. Noce, Sanford, FL (US)

(21) Appl. No.: 11/138,310

(22) Filed: May 27, 2005

(51) Int. Cl. A62B 7/10; A61M 15/08

(52) U.S. Cl. 128/206.11; 128/207.18

(57) ABSTRACT

A nose dilation device includes a resilient one-piece plastic body including a generally V-shaped center portion and a pair of substantially horizontally oriented dilation rings extending outwardly from an upper end of the center portion and adapted to internally engage nasal passages of a user.

This invention relates to nasal dilation devices and, more particularly, to a flexible resilient dilator to be inserted inside the nasal passages.

### BACKGROUND OF THE INVENTION

Nasal dilation devices designed to improve nasal breathing are well represented in the patent literature and are generally of two types. One type is externally applied and adhered on opposite sides of the nose to continually apply pulling forces to open the nasal passages. See, for example, U.S. Pat. Nos. 6,769,429; 6,375,667; 6,196,228; 6,098,616; 6,058,931; 6,029,658; 5,957,126; 5,718,224; 5,706,800; 5,653,224; 5,611,334; 5,611,333; and Re. 35,408. External nasal dilators, however, can cause general discomfort, skin irritation and discoloration.

A second type of nasal dilator is designed to be inserted into the nose and to push outwardly to open the nasal passages. See, for example, U.S. Patent Application Publication Nos. US2003/0181941 A1; US2003/0144684 A1; and U.S. Pat. No. 5,816,241. These devices may require adjustment and can also cause irritation and discomfort.

There remains a need, therefore, for a simple, inexpensive nasal dilator that is effective without causing irritation or discomfort, and which is simple in construction, easy to use and inexpensive to manufacture.

### BRIEF DESCRIPTION OF THE INVENTION

The present invention relates to a nasal dilator that is constructed of a non-invasive, flexible and resilient plastic material. The dilator is of one-piece design and is insertable and self-supporting within the nose where it exerts a gentle, outwardly-directed spring action to open the nasal passages to thereby increase the volume of air flow through the nose.

In the exemplary embodiment, the nasal dilator includes a molded plastic body having a generally V-shaped center portion and a pair of substantially horizontally oriented dilation rings extending outwardly from the upper ends of the center portion. The base of the center portion is formed with an enlarged bulbous, or rounded clamp portion that is adapted to bridge the user's septum. Angled support arms extend between the rounded clamp portion and the horizontal dilator rings. The rounded clamp portion and the angled support arms are sized and shaped to permit resilient compression of the support arms to facilitate insertion of the dilator into the user's nostrils. When the support arms are released, the dilator will expand outwardly such that the horizontal dilation rings will apply light opening pressure on the nasal passages, while the interface of the support arms and the rounded clamp portion will engage the septum and maintain the dilator in position.

The dilator rings allow air to flow through and around the device, and radiused corners at every direction-change-location on the device minimize if not eliminate irritation and thus increase the overall level of comfort. In addition, the resilient nature of the plastic material permits self-adjustment of the dilator after insertion into the nose and release of the support arms.

Accordingly, in one aspect, the invention relates to a nose dilation device comprising a resilient one-piece plastic body including a generally V-shaped center portion and a pair of substantially horizontally oriented dilation rings extending outwardly from an upper end of the center portion and adapted to internally engage nasal passages of a user.

In another aspect, the invention relates to a nose dilation device comprising a resilient one-piece plastic body including a generally V-shaped center portion and a pair of substantially horizontally oriented dilation elements extending outwardly from an upper end of the center portion and adapted to internally engage a user's nasal passage; and wherein a base of the substantially V-shaped center portion includes a rounded clamp portion adapted to externally bridge the user's septum.

In still another aspect, the invention relates to a nose dilation device comprising a septum bridge, a pair of support arms extending upwardly from the septum bridge and a dilation element extending horizontally from each support arm, wherein the support arms are resiliently compressible about the bridge to permit insertion of the support arms and the dilation elements into a user's nostrils, and resiliently expandable once inside the nostrils to open the user's nasal passages.

The invention will now be described in connection with the drawings identified below.

### BRIEF DESCRIPTION OF THE DRAWINGS

FIG. 1 is a perspective view of an exemplary embodiment of the nasal dilator;

FIG. 2 is a front elevation of the dilator shown in FIG. 1;

FIG. 3 is a top plan view of the dilator shown in FIGS. 1 and 2;

FIG. 4 is an end view of the dilator shown in FIG. 3; and

FIG. 5 is an enlarged detail taken from FIG. 4.

### DETAILED DESCRIPTION OF THE INVENTION

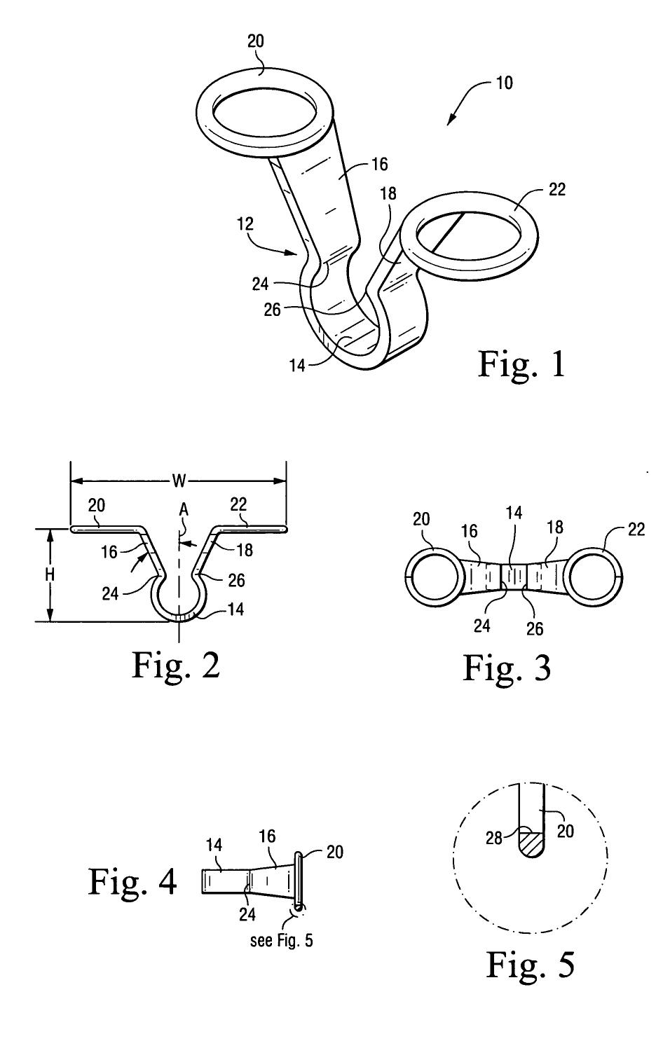

With reference to FIGS. 1-4, the nasal dilator device 10 (or simply, nasal dilator) in accordance with an exemplary embodiment, is a one-piece resilient plastic device. Nylon is especially suitable but other plastics may be effective as well. It includes a generally V-shaped center portion 12 that has a rounded or bulbous clamp portion (or septum bridge) 14 and a pair of angled support arms 16, 18 extending upwardly and outwardly therefrom. A pair of annular dilation elements 20, 22 extend substantially horizontally away from the upper ends of the respective support arms. Generally, the device may have a height H of about 0.5±0.005 in. and a width W of about 1.125±0.005 in. (see FIG. 2).

The rounded and partially annular clamp portion 14 has a curvature based on a diameter of 0.218±0.005 in., and extends about 150° where opposite ends connect at radiused interfaces 24, 26 with the support arms 16, 18, respectively. At the interfaces 24, 26, the support arms are spaced 0.125±0.005 in. from each other. The arms 16, 18 extend at about a 15° angle relative to a vertical center axis A through the device (FIG. 2). The clamp portion 14 and support arms 16, 18 have a generally rectangular cross-sectional profile as best appreciated in FIGS. 2, 3 and 4. The annular dilator rings 20, 22 each have a diameter of 0.25±0.005 in. and may have a generally round cross section. FIG. 5 illustrates a partially round cross section with a flat inner side 28 that may also be employed. The device may have a substantially uniform thickness of about 0.0312±0.005 in.

It will be appreciated that the flexible resiliency of the plastic material combined with the rounded clamp portion 14 allow the support arms to be squeezed or compressed inwardly to facilitate insertion of the device into the nose, with the clamp portion 14 externally bridging the septum. Once in place, the support arms 16, 18 are released to allow the dilator rings 20, 22 to spring outwardly to exert gentle opening pressure on the nasal passages. The resiliency and configuration of the dilator is such that the dilator rings self-adjust against the nasal passage tissue, and it will be appreciated that the dilator rings may be under about 50% compression when in place, i.e., the outward expansion of the dilator rings may be at about 50% of their unconstrained expansion.

It will also be appreciated that the dilator 10 may be manufactured in different sizes with commensurate dimensional variations in the various portions of the device.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A nose dilation device consisting of:
a resilient one-piece plastic body having a generally V-shaped center portion and a pair of substantially horizontally oriented dilation rings extending outwardly from an upper end of said center portion and adapted to internally engage nasal passages of a user.

2. The device of claim 1 wherein a base of said substantially V-shaped center portion includes a rounded clamp portion adapted to externally bridge a user's septum.

3. The device of claim 2 wherein said V-shaped center portion also includes a pair of support arms extending between said rounded clamp portion and said horizontally oriented dilation rings.

4. The device of claim 1 wherein each of said horizontally oriented dilation rings has a diameter of 0.25±0.005 in.

5. The device of claim 3 wherein said support arms extend at an angle of about 15° relative to a center axis extending perpendicular to said horizontally-oriented dilation rings.

6. The device of claim 2 wherein said rounded clamp portion has a curvature based on a diameter of 0.218±0.005 in.

7. The device of claim 3 wherein said support arms are spaced 0.125±0.005 in. from each other at a location adjacent said rounded base portion.

8. The device of claim 1 wherein said body has a thickness of about 0.0312±0.005 in.

9. The device of claim 1 wherein said body has a width of about 1.125±0.005 in.

10. The device of claim 1 wherein said body has a height of about 0.5±0.005 in.

11. The device of claim 1 wherein said resilient plastic comprises nylon.

12. A nose dilation device comprising:
a resilient one-piece plastic body consisting of a generally V-shaped center portion and a pair of substantially horizontally oriented dilation elements extending outwardly from an upper end of said center portion and adapted to internally engage a user's nasal passage; and wherein a base of said substantially V-shaped center portion includes a rounded clamp portion adapted to externally bridge the user's septum.

13. The device of claim 12 wherein said V-shaped center portion also includes a pair of support arms extending between said rounded clamp portion and said horizontally oriented dilation elements.

14. The device of claim 13 wherein said support arms extend at an angle of about 15° relative to a center axis extending perpendicular to said horizontally-oriented dilation elements.

15. The device of claim 12 wherein said rounded clamp portion has a curvature based on a diameter of 0.218±0.005 in.

16. The device of claim 13 wherein said support arms are spaced 0.125±0.005 in. from each other at a location adjacent said rounded base portion.

17. The device of claim 12 wherein said body has a thickness of about .0312±0.005 in.

18. The device of claim 12 wherein said body has a width of about 1.125±0.005 in.

19. The device of claim 12 wherein said body has a height of about 0.5±0.005 in.

20. The device of claim 12 wherein said resilient plastic comprises nylon.

21. A nose dilation device consisting of:
a septum bridge, a pair of support arms extending upwardly from said septum bridge and a dilation element extending horizontally from each support arm, wherein said support arms are resiliently compressible about said bridge to permit insertion of said support arms and said dilation elements into a user's nostrils, and resiliently expandable once inside the nostrils to open the user's nasal passages.
